(19) United States
(12) Patent Application Publication
(10) Pub. No.: US 2024/0360499 A1
(43) Pub. Date: Oct. 31, 2024

(54) GENE POLYMORPHISM MARKERS FOR DETERMINING SKIN TURNOVER AND HYPER-KERATINIZATION AND USE THEREOF

(71) Applicant: LG HOUSEHOLD & HEALTH CARE LTD., Seoul (KR)

(72) Inventors: Jiye PARK, Seoul (KR); Seung Won YOU, Seoul (KR); Yunsun KIM, Seoul (KR); Yunkwan KIM, Seoul (KR)

(73) Assignee: LG HOUSEHOLD & HEALTH CARE LTD., Seoul (KR)

(21) Appl. No.: 18/287,849

(22) PCT Filed: Apr. 27, 2022
(86) PCT No.: PCT/KR2022/005977

§ 371 Date: Oct. 20, 2023

(30) Foreign Application Priority Data
Apr. 29, 2021 (KR) 10-2021-0055764

(51) Int. Cl. C12Q 1/6858; C12Q 1/6827

(52) U.S. Cl. CPC C12Q 1/6858; C12Q 1/6827

(57) ABSTRACT

Provided are gene polymorphism markers having correlation significance with skin turnover and hyper-keratinization, a composition for determining skin turnover and hyper-keratinization, the composition including a probe capable of detecting the same or an agent capable of amplifying the same, a kit or microarray for determining skin turnover and hyper-keratinization, the kit or microarray including the composition, a method of providing information for determining skin turnover and hyper-keratinization using the gene polymorphism markers or a combination of the markers, and a method of controlling skin turnover and hyper-keratinization, the method including the steps of identifying the polymorphic site of the single nucleotide polymorphism marker and prescribing skin care products. Since the single nucleotide polymorphism markers of the present invention may provide accurate information about each individual's skin type, it is possible to develop personalized cosmetics and customized active ingredients.

### TECHNICAL FIELD

The present invention relates to gene polymorphism markers having correlation significance with skin turnover and hyper-keratinization, a composition for determining skin turnover and hyper-keratinization, the composition including a probe capable of detecting the same or an agent capable of amplifying the same, a kit or microarray for determining skin turnover and hyper-keratinization, the kit or microarray including the composition, a method of providing information for determining skin turnover and hyper-keratinization using the gene polymorphism markers or a combination of the markers, and a method of controlling skin turnover and hyper-keratinization, the method including the steps of identifying polymorphic sites of the single nucleotide polymorphism markers and prescribing skin care products.

### BACKGROUND ART

The skin is divided into three parts of epidermis, dermis, and subcutaneous fat layer from the upper layer of the skin. Among them, the epidermis consists of the basal layer, the spinous layer, the granular layer, and the cornified layer, which is the outermost layer. Epidermal cells of the basal layer are differentiated as they move up to the top of the skin, and finally reach the cornified layer. Epidermal cells that have reached the cornified layer lose their nuclei and are filled with a water-insoluble protein called keratin, turning into dead cells. The cornified layer consists of differentiated epidermal cells (keratinocytes) and skin lipids that fill the space therebetween, and prevents body substances from escaping to the outside and has a defense function to protect the human body from external physical, chemical, and biological stimuli.

When this cornified layer remains on the skin without being normally peeled off, the cornified layer becomes thick and the complexion becomes dull and dark. Impurities remaining on the skin surface or in hair follicles are oxidized or degraded by oxygen or microorganisms, and these substances cause skin troubles such as inflammation. Further, aging skin, dry skin, acne skin, etc. shows a phenomenon (stratum corneum stratification) in which the cornified layer becomes thicker because separation of the cornified layer is more delayed than normal, and the distinct feature of skin appearance is scaling of the skin. Stratum corneum stratification is mainly attributed to a decrease in the moisturizing ability of the skin, a decrease in the production and activity of desmosome (a protein that connects keratinocytes) degradation enzymes, a decrease in cell activity, etc., and is caused by skin aging, exposure to ultraviolet rays, pollution, etc.

When the cornified layer, which has been thickened by these internal and external factors, is artificially thinned by the outside, the activity or regeneration of living cells under the cornified layer is increased, which is effective in reducing the skin scales that appear on the skin surface, making the skin softer, removing wrinkles, suppressing and treating acne, etc. Therefore, many studies are being conducted to resolve stratum corneum stratification within the range that does not cause irritation.

The correlation between individual characteristics and genes is also being investigated through many studies. The chance of developing a specific disease can be predicted by diagnosing mutations, chromosomal abnormalities, etc. through genetic testing. In addition to predicting a disease, information about beauty can also be obtained. Although it is known that there are individual differences due to genetic differences, the use of standardized products that ignore these differences may hinder the perceived efficacy and satisfaction with skin improvement through use of the products. Accordingly, there is an increasing need for new methods that propose optimized management methods based on individual genetic information.

With regard to genetic diseases related to the skin barrier and keratolysis, a lot of research is being conducted on related genes, but there is little research on genes related to keratin abnormalities that cause minor symptoms or cosmetic problems, and therefore, genetic variations associated therewith are poorly understood.

### DISCLOSURE

### Technical Problem

The present inventors established a scientific skin classification standard by identifying the genetic features that determine an individual's skin characteristics, and in order to develop personalized active ingredients based on the standard and to contribute to the development of customized cosmetics for each skin characteristic through subdivision of various products, they confirmed a method of determining skin turnover and hyper-keratinization by selecting specific single nucleotide polymorphism (SNP) markers having correlation significance with skin turnover and hyper-keratinization, thereby completing the present invention.

### PRIOR ART DOCUMENTS

### Non-Patent Document

(Non-Patent Document 1) Kemp et al., Molecules. 2017 Feb. 26; 22 (3), 2017

### Technical Solution

An object of the present invention is to provide a single nucleotide polymorphism (SNP) marker for determining skin turnover and hyper-keratinization.

Another object of the present invention is to provide a composition for determining skin turnover and hyper-keratinization, the composition including a probe capable of detecting the single nucleotide polymorphism (SNP) marker for determining skin turnover and hyper-keratinization or an agent capable of amplifying the same.

Still another object of the present invention is to provide a kit or microarray for determining skin turnover and hyper-keratinization, the kit or microarray including the composition.

Still another object of the present invention is to provide a method of providing information for determining skin turnover and hyper-keratinization, the method including the step of identifying a polymorphic site of the single nucleotide polymorphism marker.

Still another object of the present invention is to provide a method of controlling skin turnover and hyper-keratinization, the method including the steps of identifying the polymorphic site of the single nucleotide polymorphism marker and prescribing skin care products.

### Advantageous Effects

Gene polymorphism markers having correlation significance with skin turnover and hyper-keratinization of the present invention may provide information about an individual's skin turnover and degree of hyper-keratinization, and furthermore, depending on information about the gene polymorphism markers observed in the individual, it will be possible to develop customized ingredients or products capable of controlling the skin turnover and hyper-keratinization.

### BRIEF DESCRIPTION OF THE DRAWING

FIG. 1 shows calculation of frequency for each genotype of rs10502560 in 348 Koreans.

FIG. 2 shows a comparison of turnover rates between genotypes of rs10502560.

FIG. 3 shows a comparison of serine prescription effects between genotypes of rs10502560.

FIG. 4 shows calculation of frequency for each genotype of rs16853334 in 348 Koreans.

FIG. 5 shows a comparison of turnover rates between genotypes of rs 16853334.

FIG. 6 shows a comparison of protease prescription effects between genotypes of rs16853334.

### BEST MODE FOR CARRYING OUT THE INVENTION

Each description and embodiment disclosed in this disclosure may also be applied to other descriptions and embodiments. That is, all combinations of various elements disclosed in this disclosure fall within the scope of the present invention. Further, the scope of the present invention is not limited by the specific description described below.

To achieve the objects of the present invention, an aspect of the present invention provides a single nucleotide polymorphism (SNP) marker for determining skin turnover and hyper-keratinization.

Another aspect of the present invention provides a composition for determining skin turnover and hyper-keratinization, the composition including a probe capable of detecting the single nucleotide polymorphism (SNP) marker for determining skin turnover and hyper-keratinization or an agent capable of amplifying the same.

As used herein, the term “polymorphism” refers to the presence of two or more alleles at one genetic locus. A variation in a single nucleotide in a polymorphic site among individuals is called single nucleotide polymorphism (SNP). Preferred polymorphism markers have two or more alleles, each occurring at frequency of 1% or more, and more specifically, of 10% or 20% or more of a selected population. The ‘gene polymorphism marker’ generally refers to a case where two or more alleles are observed at the same genetic locus (base). In general, there are major allele/major allele, major allele/minor allele, and minor allele/minor allele, depending on individuals.

In the present invention, an allele that exhibits a genetic effect on reduced turnover and increased hyper-keratinization is called an effect allele, and an allele that exhibits no genetic effect is called a non-effect allele. The effect allele may be a major allele or a minor allele, and the non-effect allele may also be a major allele or a minor allele.

Specifically, the gene polymorphism markers of the present invention have correlation significance with skin turnover and hyper-keratinization, and when one or more of the effect allele of the two alleles are possessed, it may be determined that skin turnover and hyper-keratinization are more significant, as compared to individuals with non-effect allele/non-effect allele. In other words, it is can be seen that when the non-effect allele/effect allele or the effect allele/effect allele is possessed, the skin may have characteristics of the slow turnover rate and the high degree of hyper-keratinization, as compared to the case when the non-effect allele/non-effect allele is possessed.

Since the single nucleotide polymorphism markers of the present invention allows predicting an individual's unique skin turnover and hyper-keratinization characteristics, they may also provide information on active ingredients that effectively act on the skin turnover and hyper-keratinization changes, thereby providing customized cosmetics, etc., but are not limited to thereto.

As used herein, the term “rs_id” refers to rs-ID which is an independent marker assigned to all SNPs initially registered by NCBI, which began accumulating SNP information in 1998. rs_id described in this table refers to a SNP marker, which is the polymorphism marker of the present invention.

As used herein, the term “turnover” refers to a process in which new cells made in the basal layer come up to the cornified layer and become dead cells to fall off, and new cells are born again in the basal layer. Although varying according to the region or age, the turnover period of the epidermis in the normal skin may be 4 weeks to 6 weeks. The promotion of skin turnover may be achieved by promoting the differentiation of epidermal cells, and the skin turnover may be promoted by infiltrating the basal layer and directly inducing differentiation. Further, the promotion of skin turnover may be achieved by increasing the thickness of the epidermal layer of the skin. This promotion of skin turnover may promote skin regeneration.

Further, as used herein, the term “hyper-keratinization” means that when the cornified layer is not normally peeled off and remains on the skin surface, the cornified layer becomes thicker. With respect to the objects of the present invention, the hyper-keratinization may be used interchangeably with terms such as ‘keratinization’ or ‘keratosis’.

The ‘cornified layer’ or ‘stratum corneum’ is distributed in the skin, hair, and nails and is a major component of the cytoskeleton, and consists of differentiated epidermal cells (keratinocytes) and skin lipids that fill the space therebetween, prevents the body's substances from going out of the body and performs a defense function to protect the human body from external physical, chemical and biological stimulus. Although keratinocytes are continuously produced from the inside, the old stratum corneum in the outermost layer are peeled off, maintaining a constant thickness. The epidermal cells which reached the cornified layer lose nuclei and are filled with keratin which is a water-insoluble protein, while these are converted into dead cells.

The composition of the present invention may improve hyper-keratinization by promoting the skin turnover with fewer side effects of skin irritation.

Specifically, as used herein, the skin turnover rate refers to the measurement conducted on subjects who exhibit two alleles at genetic loci (bases) of general individual non-effect allele/non-effect allele, and is obtained by quantifying the statistical correlation significance and genetic effects using linear regression analysis (corrected for age and gender). The hyper-keratinization may refer to those obtained by quantifying a desquamation index, which is a phenotype that reflects the overall amount of keratin, and a coarse flakes value, which is a phenotype that partially reflects the degree of hyper-keratinization, but is not limited thereto.

The single nucleotide polymorphism marker may be one or more single nucleotide polymorphism markers selected from single nucleotide polymorphism markers shown in Tables 1 to 3. The single nucleotide polymorphism markers shown in Tables 1 to 3 may determine whether or not having association with the skin turnover and hyper-keratinization. The skin turnover and hyper-keratinization by the single nucleotide polymorphism markers of the present invention were determined by measuring the frequency of each marker. Such significance is characterized by, but is not limited to, a p-value of less than 0.05, less than 0.01, less than 0.001, less than 0.0001, less than 0.00001, less than 0.000001, less than 0.0000001, less than 0.00000001, or less than 0.000000001. Specifically, the p-value may be less than 0.05, more specifically, the p-value may be less than 0.001, and even more specifically, less than 0.0001, but is not limited thereto.

The single nucleotide polymorphism (SNP) marker of the present invention may be one or more selected from the markers shown in Tables 1 to 3, but is not limited thereto. The single nucleotide polymorphism (SNP) marker may be one or more, and may be used in combination of two or more, three or more, four or more, etc., the combination capable of determining the skin turnover and hyper-keratinization, but is not limited thereto.

The marker may be the SNP itself, a polynucleotide consisting of 5-100 contiguous DNA sequences containing the SNP site, or a polynucleotide consisting of a complementary sequence thereof, but is not limited thereto.

Specifically, in a specific embodiment, the single nucleotide polymorphism marker may be any one or more selected from the markers shown in Table 1, but is not limited thereto.

The description of the marker selected from the markers shown in Table 1 may be as follows.

For example, when the SNP ID is rs79662935, Chr. Position (GRCh ver. 37) is described as “1:14944575” and Allele is indicated as A or G, this indicates that a base at position 14944575 of human chromosome 1 is A or G, and specifically, it means that the non-effect allele is G, and the effect allele is A.

In one specific embodiment, the marker selected from Table 1 may consist of one or more polynucleotides selected from the group consisting of a polynucleotide consisting of a 5-100 contiguous DNA sequence containing a base at position of 15130503 of human chromosome 1, which the base is G or A (rs79363155); a polynucleotide consisting of a 5-100 contiguous DNA sequence containing a base at position of 159399086 of human chromosome 2, which the base is G or A (rs12694963); a polynucleotide consisting of a 5-100 contiguous DNA sequence containing a base at position of 110989719 of human chromosome 4, which the base is A or G (rs16997129); a polynucleotide consisting of a 5-100 contiguous DNA sequence containing a base at position of 147594150 of human chromosome 5, which the base is A or G (rs79211908); a polynucleotide consisting of a 5-100 contiguous DNA sequence containing a base at position of 90525981 of human chromosome 10, which the base is G or A (rs142289305); a polynucleotide consisting of a 5-100 contiguous DNA sequence containing a base at position of 32972940 of human chromosome 12, which the base is G or A (rs1454934); a polynucleotide consisting of a 5-100 contiguous DNA sequence containing a base at position of 30256917 of human chromosome 15, which the base is G or A (rs4779686); a polynucleotide consisting of a 5-100 contiguous DNA sequence containing a base at position of 4948936 of human chromosome 16, which the base is G or A (rs12443906); a polynucleotide consisting of a 5-100 contiguous DNA sequence containing a base at position of 7977105 of human chromosome 17, which the base is A or G (rs12937410); a polynucleotide consisting of a 5-100 contiguous DNA sequence containing a base at position of 28584774 of human chromosome 18, which the base is A or C (rs8096598); a polynucleotide consisting of a 5-100 contiguous DNA sequence containing a base at position of 34136662 of human chromosome 19, which the base is A or G (rs59192758); and complementary sequences thereof, but is not limited thereto. The markers described above are only a part exemplified in Table 1, and may be selected from chromosomes at other loci in the same manner as above.

In another specific embodiment, any one or more may be selected from single nucleotide polymorphism (SNP) markers shown in Table 2, as in Table 1, but are not limited thereto.

In still another specific embodiment, any one or more may be selected from single nucleotide polymorphism (SNP) markers shown in Table 3, as in Table 1, but are not limited thereto.

The single nucleotide polymorphism markers shown in Tables 2 and 3 may be interpreted and selected as described above, but are not limited thereto.

The alleles of the present invention have the same number of chromosomes in each individual, and among them, the non-effect allele and effect allele of SNP exist, and as the base at the polymorphic site of the polymorphism marker increases by one with the effective allele, the non-effective allele may decrease by one, and as the non-effective allele increases by one, the effective allele may decrease by one. However, the range in which the effect allele and the non-effect allele may increase and decrease is within the three types of i) non-effect allele/non-effect allele, ii) non-effect allele/effect allele, iii) effect allele/effect allele, and the allele may decrease or increase within the range of the above three types, but is not limited thereto.

Further, in the present invention, the marker is a marker that may determine changes in the skin turnover and hyper-keratinization as the base of the polymorphic site of the polymorphism marker of an individual increases by one with the effect allele. More specifically, an individual possessing one or more effect alleles ((1) non-effect allele/effect allele, (2) effect allele/effect allele)) of the two alleles may be determined to have characteristics of a slow skin turnover rate and a high degree of hyper-keratinization, as compared to a general individual possessing non-effect allele/non-effect allele.

More specifically, with regard to the markers shown in Table 1, as the effect allele increases by one, it is determined that the skin turnover rate may be slow. For example, among the markers shown in Table 1, when a marker possessing the effect allele of A and the non-effect allele of G (rs79662935) in the base at position 14944575 of chromosome 1 of an individual possesses A/G or A/A, the effect size is minus, as compared to an individual possessing G/G, and thus it is determined that the skin turnover rate is slow, but is not limited thereto.

With regard to the markers shown in Table 2 or 3, as the effect allele increases by one, the increase degree of hyper-keratinization may be determined. For example, among the markers shown in Table 2, when a marker possessing the non-effect allele of A and the effect allele of G (rs184370705) in the base at position 15003662 of chromosome 1 of an individual possesses G/A or G/G, the effect size is minus (−), as compared to an individual possessing A/A, and thus it is determined that the hyper-keratinization increases, but is not limited thereto.

The above bases are exemplified only by those listed in Table 1 to Table 3, even though not described in detail, they may be interpreted and derived as described above.

As used herein, the term “probe capable of detecting the marker for determining skin turnover and hyper-keratinization” refers to a composition that may determine the skin turnover and the degree of hyper-keratinization by identifying the gene polymorphic site as described above through a specific hybridization reaction. There is no particular limitation on the specific method of such genetic analysis, and it may be any gene detection method known in the technical field to which the present invention belongs.

As used herein, the term “agent capable of amplifying the marker for determining skin turnover and hyper-keratinization” refers to a composition that may determine the skin turnover and the degree of hyper-keratinization by identifying the gene polymorphic site as described above through amplification, and specifically, refers to a primer capable of specifically amplifying the polynucleotide of the marker for determining skin turnover and hyper-keratinization.

The primer used in the amplification of the polymorphism markers refer to a single-stranded oligonucleotide that may act as a point of initiation of template-directed DNA synthesis under appropriate conditions (e.g., four different nucleoside triphosphates, and polymerase such as DNA, RNA polymerase, or reverse transcriptase) in an appropriate buffer and at an appropriate template. The appropriate length of the primer may vary depending on the purpose of use, but generally, ranges from 15 nucleotides to 30 nucleotides. Short primer molecules generally require lower temperatures to form sufficiently stable hybrid complexes with the template. A primer need not to be completely complementary to a template, but must be complementary enough to hybridize with a template.

As used herein, the term “primer” refers to a short base sequence having a free 3′ hydroxyl group that is able to form a base pair with a complementary template and serves as a starting point for copying the template strand. The primer may initiate DNA synthesis in the presence of an agent for polymerization (i.e., DNA polymerase or reverse transcriptase) and four different nucleoside triphosphates in an appropriate buffer solution at an appropriate temperature. By performing PCR amplification, the skin type may be predicted based on the production level of the desired product. PCR conditions and length of sense and antisense primers may be modified based on those known in the art.

The probe or primer of the present invention may be chemically synthesized using a phosphoramidite solid support method, or other well-known methods. These nucleic acid sequences may also be modified using many means known in the art. Non-limiting examples of such modifications include methylation, “capping”, replacement of one or more native nucleotides with analogues thereof, and inter-nucleotide modifications, for example, modifications to uncharged conjugates (e.g., methyl phosphonate, phosphotriester, phosphoroamidate, carbamate, etc.) or charged conjugates (e.g., phosphorothioate, phosphorodithioate, etc.).

Still another aspect of the present invention provides a kit for determining skin turnover and hyper-keratinization, the kit including the composition for determining skin turnover and hyper-keratinization. The kit may be an RT-PCR kit or a DNA chip kit, but is not limited thereto.

The kit of the present invention may determine the skin turnover and hyper-keratinization by identifying the SNP polymorphism markers, which are markers for determining skin turnover and hyper-keratinization, via amplification or by examining mRNA expression levels of the SNP polymorphism markers. For a specific example, in the present invention, the kit for measuring the mRNA expression levels of the markers for determining skin turnover and hyper-keratinization may be a kit including essential elements necessary for performing RT-PCR. The RT-PCR kit may include a test tube or other appropriate containers, a reaction buffer (with various pH values and magnesium concentrations), deoxynucleotides (dNTPs), enzymes such as Taq-polymerase and reverse transcriptase, DNAse, RNAse inhibitors, DEPC-water, sterile water, etc., in addition to each primer pair specific to genes of the markers for determining skin turnover and hyper-keratinization. Additionally, the kit may also include a primer pair specific to a gene used as a quantitative control. Further, the kit of the present invention may be specifically a kit for determining skin turnover and hyper-keratinization, including essential elements necessary for performing a DNA chip. The DNA chip kit is a device where a gridded array of nucleic acid species is attached to a flat solid support plate, typically, a glass surface no larger than a microscope slide, and that enables massively parallel analysis by multiple hybridization reactions between the nucleic acid on the DNA chip and the complementary nucleic acid contained in a solution treated on the surface of the DNA chip, by constantly arraying nucleic acids on the surface of the chip.

Still another aspect of the present invention provides a microarray for determining skin turnover and hyper-keratinization, the microarray including the composition for determining skin turnover and hyper-keratinization.

The microarray may include DNA or RNA polynucleotides. The microarray may be prepared in a common microarray, except that the polynucleotide of the present invention is included in the probe polynucleotide.

Methods of manufacturing a microarray by immobilizing a probe polynucleotide on a substrate are well known in the art. The probe polynucleotide, which is a hybridizable polynucleotide, refers to an oligonucleotide capable of sequence-specific binding to a complementary strand of a nucleic acid. The probe of the present invention is an allele-specific probe, where a polymorphic site is present in a nucleic acid fragment derived from two members of the same species and hybridizes to a DNA fragment derived from one member but not to a DNA fragment derived from the other member. In this case, the hybridization conditions show a significant difference in hybridization intensity between alleles, and must be stringent enough to hybridize to only one of the alleles. By this way, good hybridization differences between different allelic forms may be induced. The probe of the present invention may be used for determining skin turnover and hyper-keratinization by detecting alleles, etc. The determination methods may include detection methods based on hybridization of nucleic acids such as southern blotting, etc., and may be provided in a form already bound to a substrate of a DNA chip in a method of using the DNA chip. The hybridization may be commonly performed under stringent conditions, e.g., at a salt concentration of 1 M or less and a temperature of 25° C. or higher. For example, conditions of 5×SSPE (750 mM NaCl, 50 mM Na Phosphate, 5 mM EDTA, pH 7.4) and 25° C. to 30° C. may be suitable for allele-specific probe hybridization.

The process of immobilizing a probe polynucleotide associated with the determination of skin turnover and hyper-keratinization of the present invention on a substrate may also be easily performed using such an existing technique. Further, the hybridization of nucleic acids on a microarray and detection of hybridization results are well known in the art. With regard to the detection, hybridization results may be detected, for example, by labeling a nucleic acid sample with a labeling material capable of generating a detectable signal including a fluorescent material, such as Cy3 and Cy5, hybridizing on a microarray, and detecting the signal generated from the labeled material.

Still another aspect of the present invention provides a method of providing information for determining skin turnover and hyper-keratinization, the method including the steps of: (a) amplifying the polymorphic site of the single nucleotide polymorphism marker in DNA obtained from a sample isolated from a subject or hybridizing the same with a probe; and (b) identifying the nucleotide sequence of the amplified or hybridized polymorphic site of the step (a).

As used herein, the term “subject” refers to a test subject, of which the skin turnover and hyper-keratinization are intended to determine. DNA may be obtained from a sample such as hair, urine, blood, various body fluids, isolated tissues, isolated cells, or saliva, etc., but is not limited thereto.

The method of obtaining genomic DNA of the step (a) may be performed by using any method known to those skilled in the art.

The step of amplifying the polymorphic site of the single nucleotide polymorphism marker from the obtained DNA or hybridizing the same with a probe in the step (a) may be performed by using any method known to those skilled in the art. For example, the target nucleic acid may be obtained by amplifying through PCR and purifying. In addition, ligase chain reaction (LCR) (Wu and Wallace, Genomics 4, 560 (1989), Landegren et al., Science 241, 1077 (1988)), transcription amplification (Kwoh et al., Proc. Natl. Acad. Sci. USA 86, 1173 (1989)), self-sustained sequence replication (Guatelli et al., Proc. Natl. Acad. Sci. USA 87, 1874 (1990)), and nucleic acid based sequence amplification (NABSA), etc. may be used.

In the method, the determination of the nucleotides of the polymorphic site of the step (b) may be performed by sequencing analysis, hybridization by microarray, allele-specific PCR, dynamic allele-specific hybridization (DASH), PCR extension analysis, SSCP, PCR-RFLP analysis or TaqMan technique, SNPlex platform (Applied Biosystems), mass spectrometry (e.g., MassRAY system of Sequenom), mini-sequencing, Bio-Plex system (BioRad), CEQ and SNPstream system (Beckman), Molecular Inversion Probe array technique (e.g., Affymetrix GeneChip), and BeadArray Technologies (e.g., Illumina GoldenGate and Infinium assay), but is not limited thereto. By the above methods or other methods available to those skilled in the art to which the present invention pertains, one or more alleles in the polymorphism marker, including microsatellites, SNPs or other types of polymorphism markers, may be identified. Such determination of the base of the polymorphic site may be specifically performed through a SNP chip.

The method may further include (c) determining that the skin turnover rate is slow or hyper-keratinization is increased when the base of the amplified or hybridized polymorphic site includes one or more of the bases that are the effect alleles according to the single nucleotide polymorphism markers, but is not limited to thereto.

As used herein, the term “SNP chip” refers to one of DNA microarrays capable of identifying each base of hundreds of thousands of SNPs at once.

The TaqMan method includes (1) the step of designing and preparing primers and TaqMan probes to amplify a desired DNA fragment; (2) the step of labeling different probes of alleles with FAM dye and VIC dye (Applied Biosystems); (3) the step of performing PCR using the DNA as a template and using the primers and probes; (4) the step of analyzing and confirming a TaqMan assay plate with a nucleic acid analyzer, after the above PCR reaction is completed; and (5) the step of determining the genotype of the polynucleotide of the step (1) from the analysis results.

In the above, the sequencing analysis may be performed using a common method of determining a base sequence, or may be performed using an automated genetic analyzer. In addition, allele-specific PCR refers to a PCR method that amplifies a DNA fragment where the SNP is located using a primer set including primers designed with the bases where the SNP is located as the 3′-end. A principle of the method is that, for example, when a specific base is substituted from A to G, if PCR is performed by designing a primer containing A as the 3′ terminal base and a reverse primer capable of amplifying an appropriate size of a DNA fragment, when the base at the SNP site is A, amplification reaction is normally performed and a band at the desired position is observed, and when the base is replaced by G, the primer is able to complementarily bind to the template DNA, but the amplification reaction is not properly performed due to the failure of complementary bonds at the 3′-end. DASH may be performed by common methods, and specifically, by a method by Prince et al.

Meanwhile, in the PCR extension analysis, a DNA fragment containing the base where the single nucleotide polymorphism is located is first amplified with a primer pair, and then all nucleotides added to the reaction are inactivated by dephosphorylation, and SNP-specific extension primers, a dNTP mixture, dideoxynucleotides, a reaction buffer, and DNA polymerase are added thereto to perform a primer extension reaction. With regard to the extension primers, a base immediately adjacent to the 5′ direction of the base where the SNP is located is used as the 3′-end. In the dNTP mixture, nucleic acids having the same base as the dideoxynucleotide are excluded, and the dideoxynucleotide is selected from one of the base types representing the SNP. For example, when there is a substitution from A to G and a mixture of dGTP, dCTP and dTTP, and ddATP are added to the reaction, the primer is extended by DNA polymerase at the base where the substitution occurred, and after a few bases, and the primer extension reaction is terminated by ddATP at the position where the base A first appears. When the substitution has not occurred, the extension reaction is terminated at that position, and thus the type of base representing the SNP may be determined by comparing the length of the extended primer.

With regard to the detection method, the SNP may be detected by detecting fluorescence using a genetic analyzer (e.g., ABI's Model 3700, etc.) which is used for general base sequence determination, when the extension primers or dideoxynucleotides are tagged with fluorescence labels. When unlabeled extension primers and dideoxynucleotides are used, the SNP may be detected by measuring a molecular weight using matrix assisted laser desorption ionization-time of flight (MALDI-TOF) technique.

Still another aspect of the present invention provides a method of controlling skin turnover and hyper-keratinization, the method including the steps of (a) amplifying the polymorphic site of the single nucleotide polymorphism marker in DNA obtained from a sample isolated from a subject or hybridizing the same with a probe; (b) identifying the nucleotide sequence of the amplified or hybridized polymorphic site of the step (a); and (c) prescribing skin care products when the base of the amplified or hybridized polymorphic site includes one or more of the bases that are the effect alleles according to the single nucleotide polymorphism markers.

The step (a) and the step (b) are as described above.

Additionally, provided is a method of controlling skin turnover and hyper-keratinization by (c) prescribing skin care products when the base of the amplified or hybridized polymorphic site includes one or more of the bases that are the effect alleles according to the single nucleotide polymorphism markers, but is not limited thereto.

For example, among the markers shown in Table 1, in the case where the non-effect allele is A and the effect allele is G (rs10502560) in the base at position 28732877 of chromosome 18 of an individual, the skin turnover rate was significantly slow in those possessing G/A or G/G, as compared with those possessing A/A. It was confirmed that when skin care products were prescribed to improve the skin turnover rate, the skin turnover rate was significantly increased in the groups including the effect allele.

The above skin care products are not limited as long as they may affect the control of the skin turnover and hyper-keratinization. For example, they may be serine or protease, but are not limited thereto.

### MODE FOR CARRYING OUT THE INVENTION

Hereinafter, the present invention will be described in more detail with reference to exemplary embodiments. However, these exemplary embodiments are only for illustrating the present invention, and the scope of the present invention is not intended to be limited by these exemplary embodiments.

It was intended to discover genomic sites (gene mutations) that show differences in the facial skin turnover rate and the degree of hyper-keratinization (value) according to genetic information in Koreans. The present invention is intended to discover genomic sites (gene mutations) associated with skin turnover and hyper-keratinization, and a microarray genotyping chip (a product of Illumina) capable of screening the whole genomic level without pre-selecting gene candidates was used.

In the present invention, a method of acquiring the phenotype of Experimental Examples below was used in order to evaluate the skin turnover and the degree of hyper-keratinization, and used after correction for age and BMI, cluster principal components in order to minimize external effects that may affect skin turnover and hyper-keratinization (values).

In order to confirm the association between genomic sites (gene mutations) and skin turnover and hyper-keratinization (values), linear regression analysis was used to quantify correlation significance and genetic effects.

### Experimental Example 1: Acquisition of Phenotype Information

### Experimental Example 1-1: Skin Turnover Rate

By referring to a DHA staining method (PLOS One. 2019 Apr. 16; 14 (4): e0215244), which is an existing method of measuring skin turnover rates, a new method of measuring the skin turnover rates was designed by modifying and changing the DHA staining method. Instead of the existing DHA, a genipin component of Genipa americana extract was used, which reacts with amine groups of proteins of the cornified layer, showing a dark blue color (Book Ethnobotany: Application of Medicinal Plants chapter: Ethnobotanical Retrospective and Features of the Multipurpose Plant Genipa americana L.).
1) The inner side of the upper arm where the skin turnover rate will be measured is prepared by alcohol swabbing.
2) The original skin L (L0) value of the area to be dyed is measured (using a colorimeter CR-400, Konica Minolta).
3) The inner side of the upper arm is dyed by applying 40 μL of a dye (commercially available product containing G. Americana extract) using a patch test unit (IQ Ultra™).
4) After 1 hour of dyeing, the patch test unit is removed and the residual dye is rinsed off with running water.
5) The initial L (L1) value of the dyed area is measured on the 3rd day of dyeing to ensure sufficient color development (artificial desquamation is prevented and controlled by prohibiting the use of cleansers, body wash, shower balls, etc., on the dyed area after dyeing).
6) The difference between L0 and L1 is considered 100%, and the L values of the dyed area are measured every 3 days at the same time until the L value after dyeing reaches 50% (although differences may occur during the measurement period due to the difference in the turnover rate between individuals, measurements are made for approximately 3 weeks, and a linear L value change is usually observed).
7) When the measurement is completed, the rate of change in the L values is calculated using the first measurement L (L1) value and the last measurement L (L2) value (L2−L1)/(number of days between the first measurement date and the last measurement date)=(dL/day).
8) The rate of change in the L values (dL/day) is defined as the skin turnover rate.

### Experimental Example 1-2: Desquamation Index

To indicate the overall quantity of stratum corneum, desquamation index, which is one of the existing indicators for measuring stratum corneum, was determined using Visioscan VC 98 (Courage & Khazaka Electronic GmbH) and D-squame® (Cu derm) (J Cosmet Dermatol. 2020 October; 19 (10): 2606-2615., J Clin Aesthet Dermatol. 2020 August; 13 (8): E54-E58).
1) Referring to the previously known method, D-squame® is applied to both cheeks using a transparent stratum corneum tape D-squame® (Cu derm) and a dedicated pressure device (Cu Derm), and pressed down for 5 seconds and removed.
2) D-squame® is placed on black paper and images of stratum corneum removed from both cheeks are obtained using Visioscan VC98 (when a thick layer is formed due to a lot of stratum corneum, it appears white because the irradiated UVA cannot pass through it, and when a thin layer is formed due to little stratum corneum, it appears black).
3) The desquamation index (program-calculated value) is determined using SELS which is a measurement program of Visioscan VC98.

The pixels of the images which have been converted to 254 gray scale depending on the thickness of the stratum corneum are classified into five stages based on color (=according to thickness). Weighted values are assigned to each stage, and the desquamation index is calculated using the total stratum corneum area (%) and the stratum corneum area (%) of each stage. This index may be used as an indicator of the total amount of stratum corneum by putting high weights to stratum corneum of the thick layer and low weights to stratum corneum of the thin layer.

Calculation formula: Desquamation Index=(2A+Σ[Tn×(n−1)])/6

A=total stratum corneum area, Tn=stratum corneum area of each stage, n=1˜5 (stage 5 is the thickest stratum corneum)

### Experimental Example 1-3: Coarse Flakes Value

To indicate partial hyper-keratinization, the coarse flakes value was defined and determined by referring to and modifying the desquamation index calculation formula using Visioscan VC98 (Courage & Khazaka Electronic GmbH) and D-squame® (Cu derm).
1) Referring to the previously known method, D-squame® is applied to both cheeks using a transparent stratum corneum tape D-squame® (Cu derm) and a dedicated pressure device (Cu Derm), and pressed down for 5 seconds and removed.
2) D-squame® is placed on black paper and images of stratum corneum removed from both cheeks are obtained using Visioscan VC98 (when a thick layer is formed due to a lot of stratum corneum, it appears white because the irradiated UVA cannot pass through it, and when a thin layer is formed due to little stratum corneum, it appears black).
3) The coarse flakes value (program-calculated value) is determined using SELS which is a measurement program of Visioscan VC98.

The pixels of the images which have been converted to 254 gray scale depending on the thickness of the stratum corneum are classified into five stages based on color (=according to thickness). The thin stratum comeum at stages 1, 2, and 3 are classified as fine flakes, and the thick stratum corneum at stages 4 and 5 are classified as coarse flakes.

$$Calculation formula : Coarse flakes value = ( T 4 + T 5 ) / A * 100$$

$$A = total corneum area , T 4 = stratum corneum area at stage 4 , T 5 = stratum corneum area at stage 5$$

### Experimental Example 2: Gene Collection

To derive general gene polymorphism markers that may explain skin turnover and hyper-keratinization, healthy Koreans in their 20s to 60s were recruited.

Gene collection was conducted through saliva collection, and for effective gene collection, all subjects of analysis were prohibited from consuming any food, including water, from 30 minutes before collection.

From the above subjects, excluded were 1) subjects who are pregnant, lactating, or planning to become pregnant within 6 months, 2) subjects who have used steroid-containing skin preparations for 1 month or more in order to treat skin diseases, 3 6 months have not passed since participating in the same test, 4 subjects who have sensitive or hypersensitive skin, 5 subjects who have skin abnormalities such as spots, acne, erythema, or telangiectasia on the test area, 6 subjects who used the same or similar cosmetics or medicines on the test area within 3 months of starting the test, 7 subjects who have received a procedure (skin peeling, Botox, other skin care) on the test area or plan to receive the same procedure within 6 months, 8 subjects who have a chronic wasting disease (asthma, diabetes, high blood pressure, etc.), 9 subjects who have atopic dermatitis, and 10 other subjects who are considered to have difficulty with the test by a principal investigator.

### Experimental Example 3: Genotype Analysis According to Skin Characteristics

For gene extraction from saliva for genetic analysis, human genomic DNA was extracted using a QIAamp mini prep kit (QIAGEN), and its quality was confirmed through a band examination by absorbance (OD 260/280) or 1.7, a concentration of 50 ng/ul, 1× TAE 1% agarose gel, and genetic analysis was performed only on those that passed the quality test.

Genetic analysis was conducted using an Illumina microarray genotyping chip, and specifically, the genes of the subjects were analyzed using a global screening array product from the same company.

The genetic analysis experiment using the Illumina microarray genotyping chip was conducted according to the provided manual, and the provided reagents were used to perform procedures of genomic DNA amplification, DNA fragmentation, precipitation, hybridization, staining, washing, coating, and scanning.

The microarray genotyping chip on which the experiment was completed was scanned using iScan Control Software (Illumina). Once scanning was completed, an idat file was automatically created, and data quality control and genetic information confirmation were performed using a Plink program. Specifically, standards of the above data quality control are sample call rate >95%, marker call rate >95% for phenotype 1 (turnover rate), and sample call rate >90%, marker call rate >95% for phenotype 2 (desquamation index) or phenotype 3 (coarse flakes value).

In this experiment, only data that passed the data quality control after genetic analysis were used.

### Experimental Example 4: Extraction of Gene Polymorphism Markers with Correlation Significance with Skin Turnover and Hyper-Keratinization

For quality control of the gene polymorphism markers to be analyzed, only gene polymorphism markers exceeding the effect allele frequency or 0.05 and Hardy-Weinberg equilibrium or 0.000001 were used.

Specifically, with regard to phenotype 1 (skin turnover), the correlation between the skin turnover rate and gene SNPs was examined in 34 people, and with regard to phenotype 2 (desquamation index) and phenotype 3 (coarse flakes value), the correlation between the desquamation index, coarse flakes value, and gene SNPs was examined in 176 people (p<0.05).

Statistical correlation significance and genetic effects were quantified using linear regression analysis (corrected for age and gender).

As the effect allele of the selected SNP is increased by one, the degree of increase or decrease in the phenotype was defined as the effect size.

y˜β_1 x_1+β_2 x_2+β_3 x_3

(y: phenotype, β_1: effect size of age, β_2: effect size of gender, β_3: effect size of genotype, x_1: age, x_2: gender, x_3: genotype)

The lists of SNP markers significantly associated with skin turnover and hyper-keratinization are shown in Table 1 to Table 3 below. Specifically, the gene polymorphism markers associated with skin turnover, with a significance level (P) of less than 0.05, are shown in Table 1. Further, the gene polymorphism markers associated with the desquamation index which is related to skin hyper-keratinization are shown in Table 2. The gene polymorphism markers associated with coarse flakes value which is related to skin hyper-keratinization, with a significance level (P) of less than 0.05, are shown in Table 3.

TABLE 1
Gene polymorphism markers associated with skin turnover (P < 0.05)

| | | | | | | Effect | Non-effect | p- | Effect | | | | |
|---|---|---|---|---|---|---|---|---|---|---|---|---|---|
| No. | SNP_rs id | Chromosome | Position | Gene | Consequence | allele | allele | value | size | EAF_KOR | EAF_CHN | EAF_JPA | EAF_EUR |
| 1 | rs79662935 | 1 | 14944575 | KAZN | intron_variant | A | G | 0.02603904 | 0.999532251 | 0.97 | 0.93 | 0.97 | 0.98 |
| 2 | rs79363155 | 1 | 15130503 | KAZN | intron_variant | G | A | 0.02603904 | −0.999532251 | 1 | 1 | 1 | 0.94 |
| 3 | rs12739376 | 1 | 15262708 | KAZN | intron_variant | G | A | 0.032233581 | −0.519443628 | 0.91 | 0.84 | 0.91 | 0.86 |
| 4 | rs12409399 | 1 | 15317488 | KAZN | intron_variant | A | G | 0.006493726 | −0.294233284 | 0.74 | 0.7 | 0.81 | 0.72 |
| 5 | rs12694963 | 2 | 159399086 | PKP4 | intron_variant | G | A | 0.025187873 | −1.00084276 | 0.98 | 0.99 | 0.99 | 0.98 |
| 6 | rs116232130 | 2 | 159422972 | PKP4 | intron_variant | A | G | 0.02603904 | −0.999532251 | 0.97 | 0.99 | 0.98 | 0.99 |
| 7 | rs2214903 | 2 | 159429406 | PKP4 | intron_variant | A | G | 0.015260999 | −0.259457727 | 0.38 | 0.34 | 0.49 | 0.59 |
| 8 | rs13384591 | 2 | 159500241 | PKP4 | intron_variant | G | A | 0.019126418 | −0.284056324 | 0.26 | 0.17 | 0.21 | 0.32 |
| 9 | rs4673920 | 2 | 215810303 | ABCA12 | intron_variant | G | A | 0.040811241 | −0.229574502 | 0.65 | 0.66 | 0.61 | 0.61 |
| 10 | rs16853334 | 2 | 215951405 | ABCA12 | intron_variant | G | A | 0.04606757 | −0.20504794 | 0.49 | 0.6 | 0.5 | 0.91 |
| 11 | rs16997129 | 4 | 110989719 | ELOVL6 | intron_variant | A | G | 0.020738708 | −0.292564934 | 0.76 | 0.7 | 0.76 | 0.2 |
| 12 | rs6533491 | 4 | 111030787 | ELOVL6 | intron_variant | G | A | 0.006429482 | −0.422659799 | 0.88 | 0.8 | 0.84 | 0.53 |
| 13 | rs79211908 | 5 | 147594150 | SPINK6 | intron_variant | A | G | 0.037701317 | −0.231410839 | 0.8 | 0.82 | 0.82 | 0.94 |
| 14 | rs142289305 | 10 | 90525981 | LIPN | intron_variant | G | A | 0.018356855 | −0.676588025 | 0.98 | 0.98 | 1 | 1 |
| 15 | rs402781 | 10 | 90526379 | LIPN | intron_variant | G | A | 0.026880656 | −0.38717037 | 0.88 | 0.9 | 0.93 | 0.82 |
| 16 | rs1454934 | 12 | 32972940 | PKP2 | intron_variant | G | A | 0.000585272 | −0.607837443 | 0.89 | 0.9 | 0.89 | 0.85 |
| 17 | rs1137015 | 12 | 52702681 | KRT86 | 3_prime_UTR_variant | G | A | 0.02106493 | 0.362818462 | 0.88 | 0.9 | 0.92 | 0.87 |
| 18 | rs638043 | 12 | 53044267 | KRT2 | missense_variant | G | A | 0.015474419 | −0.64607615 | 0.98 | 0.97 | 0.98 | 0.84 |
| 19 | rs10783528 | 12 | 53088484 | KRT77 | missense_variant | G | A | 0.007017946 | −0.315402298 | 0.37 | 0.43 | 0.34 | 0.64 |
| 20 | rs4919750 | 12 | 53187150 | KRT3 | intron_variant | G | A | 0.022235212 | −0.402930105 | 0.13 | 0.08 | 0.12 | 0.18 |
| 21 | rs2307027 | 12 | 53204614 | KRT4 | intron_variant | G | A | 0.016464241 | −0.35708733 | 0.17 | 10.14 | 0.17 | 0.15 |
| 22 | rs72648108 | 12 | 53293388 | KRT8 | intron_variant | A | C | 0.040541637 | −0.321860508 | 0.16 | 0.15 | 0.2 | 0.01 |
| 23 | rs4779686 | 15 | 30256917 | TJP1 | intron_variant | G | A | 0.039057432 | −0.225152705 | 0.56 | 0.6 | 0.61 | 0.79 |
| 24 | rs11247210 | 15 | 101039159 | CERS3 | intron_variant | G | A | 0.030075896 | −0.245412924 | 0.4 | 0.42 | 0.38 | 0.52 |
| 25 | rs66846923 | 15 | 101939756 | PCSK6 | intron_variant | A | G | 0.033230043 | −0.357440219 | 0.21 | 0.23 | 0.2 | 0.06 |
| 26 | rs7172235 | 15 | 101955037 | PCSK6 | intron_variant | A | G | 0.026995068 | −0.408983825 | 0.81 | 0.84 | 0.79 | 0.73 |
| 27 | rs7168655 | 15 | 101964921 | PCSK6 | intron_variant | A | G | 0.00320997 | −0.45064594 | 0.15 | 0.14 | 0.25 | 0.37 |
| 28 | rs113379051 | 15 | 101968465 | PCSK6 | intron_variant | A | G | 0.040817577 | −0.546311446 | 0.02 | 0.01 | 0.02 | 0.09 |
| 29 | rs76959991 | 15 | 101970284 | PCSK6 | intron_variant | G | A | 0.016341885 | −0.519935918 | 0.93 | 0.94 | 0.93 | 0.97 |
| 30 | rs12594107 | 15 | 101990581 | PCSK6 | intron_variant | A | G | 0.006805838 | −0.438711247 | 0.18 | 0.14 | 0.17 | 0.37 |
| 31 | rs7172696 | 15 | 101991311 | PCSK6 | intron_variant | G | A | 0.028971904 | −0.256719769 | 0.42 | 0.37 | 0.46 | 0.69 |
| 32 | rs903551 | 15 | 101997936 | PCSK6 | intron_variant | A | G | 0.047701874 | −0.215188396 | 0.57 | 0.59 | 0.42 | 0.84 |
| 33 | rs12443906 | 16 | 4948936 | PPL | intron_variant | G | A | 0.023848153 | −0.309793291 | 0.28 | 0.31 | 0.27 | 0.28 |
| 34 | rs12937410 | 17 | 7977105 | ALOX12B | intron_variant | A | G | 0.04075334 | −0.352395056 | 0.16 | 0.2 | 0.12 | 0.21 |
| 35 | rs11653427 | 17 | 39620262 | KRT32 | intron_variant | A | G | 0.030894141 | −0.25252801 | 0.32 | 0.31 | 0.39 | 0.42 |
| 36 | rs1003842 | 17 | 39646021 | KRT36 | synonymous_variant | A | G | 0.034880457 | −0.294826021 | 0.26 | 0.26 | 0.28 | 0.29 |
| 37 | rs7406225 | 17 | 39920257 | JUP | intron_variant | G | A | 0.004889038 | −0.319226653 | 0.47 | 0.48 | 0.45 | 0.72 |
| 38 | rs8096598 | 18 | 28584774 | DSC3 | intron_variant | A | C | 0.013088352 | −0.471106812 | 0.87 | 0.81 | 0.87 | 0.93 |
| 39 | rs17799745 | 18 | 28666826 | DSC2 | intron_variant | A | G | 0.046896406 | −0.235764161 | 0.83 | 0.81 | 0.83 | 0.88 |
| 40 | rs10502560 | 18 | 28732877 | DSC1 | intron_variant | G | A | 0.034555556 | −0.240591868 | 0.39 | 0.43 | 0.35 | 0.79 |
| 41 | rs59192758 | 19 | 34136662 | CHST8 | intron_variant | A | G | 0.004015304 | −0.931205884 | 0.96 | 0.94 | 0.95 | 1 |
| 42 | rs8106048 | 19 | 34182143 | CHST8 | intron_variant | G | A | 0.045675033 | −0.432317625 | 0.13 | 0.21 | 0.15 | 0.15 |
| 43 | rs11084746 | 19 | 34183593 | CHST8 | intron_variant | A | G | 0.045675033 | −0.432317625 | 0.13 | 0.2 | 0.14 | 0.11 |
| 44 | rs2041726 | 19 | 34189387 | CHST8 | intron_variant | C | A | 0.029319686 | −0.362743234 | 0.89 | 0.88 | 0.87 | 0.86 |
| 45 | rs2041727 | 19 | 34189479 | CHST8 | intron_variant | G | A | 0.029319686 | −0.362743234 | 0.89 | 0.88 | 0.87 | 0.86 |
| 46 | rs2041728 | 19 | 34189637 | CHST8 | intron_variant | G | A | 0.029319686 | −0.362743234 | 0.89 | 0.86 | 0.87 | 0.86 |
| 47 | rs2041729 | 19 | 34189756 | CHST8 | intron_variant | G | A | 0.029319686 | −0.362743234 | 0.89 | 0.86 | 0.87 | 0.86 |
| 48 | rs2041730 | 19 | 34189766 | CHST8 | intron_variant | A | G | 0.029319686 | −0.362743234 | 0.89 | 0.86 | 0.87 | 0.86 |
| 49 | rs7254433 | 19 | 34189956 | CHST8 | intron_variant | G | A | 0.029319686 | −0.362743234 | 0.89 | 0.88 | 0.87 | 0.86 |
| 50 | rs76630270 | 19 | 34197923 | CHST8 | intron_variant | A | G | 0.02476765 | −0.536910334 | 0.09 | 0.15 | 0.08 | 0.06 |
| 51 | rs6510493 | 19 | 35997457 | DMKN | intron_variant | A | C | 0.026441963 | −0.370891865 | 0.18 | 0.24 | 0.22 | 0.19 |

TABLE 2
Gene polymorphism markers associated with desquamation index related to skin hyper-keratinization (P < 0.05)

| | | | | | | Effect | Non-effect | | Effect | | EAF_CH | | |
|---|---|---|---|---|---|---|---|---|---|---|---|---|---|
| No. | SNP_rs id | Chromosome | Position | Gene | Consequence | allele | allele | p-value | size | EAF_KOR | N | EAF_JPN | EAF_EUR |
| 1 | rs184370705 | 1 | 15003662 | KAZN | intron_variant | G | A | 0.034888977 | 4.491138758 | 0 | 0.02 | 0 | 0 |
| 2 | rs3765381 | 1 | 15190029 | KAZN | intron_variant | A | G | 0.03287133 | 1.115724854 | 0.04 | 0.06 | 0.14 | 0.13 |
| 3 | rs16851179 | 1 | 15377086 | KAZN | intron_variant | G | A | 0.04331357 | 1.47991479 | 0.03 | 0.04 | 0.01 | 0.05 |
| 4 | rs6695209 | 1 | 15430895 | KAZN | intron_variant | G | A | 0.01323609 | 1.636944119 | 0.96 | 0.93 | 0.99 | 0.84 |
| 5 | rs147613842 | 1 | 152280857 | FLG | missense_variant | G | A | 0.028827128 | 1.242932251 | 0.05 | 0.05 | 0.05 | 0 |
| 6 | rs2193707 | 2 | 159320951 | PKP4 | intron_variant | G | A | 0.048194815 | 0.568366856 | 0.15 | 0.12 | 0.18 | 0.44 |
| 7 | rs3771595 | 2 | 159393449 | PKP4 | intron_variant | G | A | 0.032661796 | 0.55315233 | 0.21 | 0.22 | 0.22 | 0.3 |
| 8 | rs2214903 | 2 | 159429406 | PKP4 | intron_variant | A | G | 0.028499452 | 0.49437105 | 0.38 | 0.34 | 0.49 | 0.59 |
| 9 | rs13384591 | 2 | 159500241 | PKP4 | intron_variant | G | A | 0.012214112 | 0.621639471 | 0.26 | 0.17 | 0.21 | 0.32 |
| 10 | rs2030628 | 2 | 215922612 | ABCA12 | intron_variant | G | A | 0.034041577 | 0.619503423 | 0.22 | 0.29 | 0.28 | 0.39 |
| 11 | rs13414595 | 2 | 215949200 | ABCA12 | intron_variant | A | C | 0.04586506 | 0.592248471 | 0.2 | 0.27 | 0.27 | 0.54 |
| 12 | rs3132555 | 6 | 31082910 | CDSN | intron_variant | G | C | 0.035576401 | 0.69396991 | 0.11 | 0.89 | 0.86 | 0.74 |
| 13 | rs3132552 | 6 | 31085269 | CDSN | intron_variant | A | G | 0.035576401 | 0.69396991 | 0.11 | 0.11 | 0.14 | 0.26 |
| 14 | rs9263670 | 6 | 31087295 | CDSN | intron_variant | G | C | 0.035576401 | 0.69396991 | 0.11 | 0.89 | 0.86 | 0.74 |
| 15 | rs666769 | 6 | 13841587 | PERP | intron_variant | G | A | 0.006249976 | 1.046351636 | 0.07 | 0.05 | 0.12 | 0.34 |
| 16 | rs117579710 | 11 | 130066788 | ST14 | intron_variant | A | G | 0.014513862 | 5.19865694 | 0 | 0 | 0 | 0.04 |
| 17 | rs7304398 | 12 | 3300002 | PKP2 | intron_variant | A | G | 0.013933191 | 0.7645445 | 0.81 | 0.8 | 0.83 | 0.9 |
| 18 | rs76774152 | 12 | 33010856 | PKP2 | intron_variant | A | G | 0.011892681 | 0.621999699 | 0.27 | 0.27 | 0.29 | 0.03 |
| 19 | rs7264819 | 12 | 53315578 | KRT8 | intron_variant | G | A | 0.012361163 | 1.419512976 | 0.96 | 0.96 | 0.98 | 1 |
| 20 | rs35312232 | 14 | 24724663 | TGM1 | synonymous_variant | G | A | 0.04759288 | 4.22188268 | 1 | 1 | 1 | 0.99 |
| 21 | rs7742881717 | 15 | 100940908908 | CERS3S3 | 3_prime_UTR_V_UTR_V | G | A | 0.0431898059805 | 1.4791367766776 | 0.98 | 0.97 | 0.96 | 0.97 |
| 22 | rs8028803 | 15 | 101008670 | CERS3 | intron_variant | A | G | 0.004282997 | 0.641926497 | 0.41 | 0.43 | 0.36 | 0.52 |
| 23 | rs12916756 | 15 | 101010137 | CERS3 | intron_variant | G | A | 0.004282997 | 0.641926497 | 0.41 | 0.43 | 0.36 | 0.51 |
| 24 | rs11247210 | 15 | 101039159 | CERS3 | intron_variant | G | A | 0.04074673 | 0.469724102 | 0.4 | 0.42 | 0.38 | 0.52 |
| 25 | rs11247218 | 15 | 101073150 | CERS3 | intron_variant | A | G | 0.010898117 | 0.631545081 | 0.73 | 0.71 | 0.6 | 0.46 |
| 26 | rs7172235 | 15 | 10195537 | PCSK6 | intron_variant | A | G | 0.03601626 | 0.57147454 | 0.81 | 0.84 | 0.79 | 0.73 |
| 27 | rs7169026 | 15 | 101965121 | PCSK6 | intron_variant | G | A | 0.043927532 | 0.546523294 | 0.79 | 0.83 | 0.8 | 0.73 |
| 28 | rs12903048 | 15 | 102006325 | PCSK6 | intron_variant | C | A | 0.029698484 | 0.484549718 | 0.45 | 0.44 | 0.53 | 0.21 |
| 29 | rs7166898 | 15 | 10202148 | PCSK6 | intron_variant | A | G | 0.043494003 | 0.477741834 | 0.32 | 0.32 | 0.47 | 0.12 |
| 30 | rs3027300 | 17 | 7988993 | ALOX12B | intron_variant | A | C | 0.0345137 | 0.565947386 | 0.8 | 0.86 | 0.83 | 0.85 |
| 31 | rs3027215 | 17 | 8018506 | ALOXE3 | intron_variant | A | G | 0.0214569 | 1.15426449 | 0.05 | 0.05 | 0.04 | 0.09 |
| 32 | rs146438034 | 17 | 39091664 | KRT23 | intron_variant | A | G | 0.025925424 | 2.749189808 | 0.99 | 1 | 0.97 | 1 |
| 33 | rs1736442 | 18 | 55225777 | FECH | intron_variant | A | G | 0.036026029 | 0.495367942 | 0.36 | 0.42 | 0.36 | 0.98 |
| 34 | rs16968313 | 19 | 34131564 | CHST8 | intron_variant | A | C | 0.045465681 | 0.708978781 | 0.91 | 0.91 | 0.91 | 0.93 |
| 35 | rs74588143 | 19 | 34151523 | CHST8 | intron_variant | G | A | 0.008249574 | 1.387426959 | 0.94 | 0.96 | 0.93 | 1 |
| 36 | rs4806281 1 | 19 | 36637393 | CAPNS1 | intron_variant | A | G | 0.02016886 | 0.927637086 | 0.08 | 0.06 | 0.08 | 0.02 |

TABLE 3
Gene polymorphism markers associated with coarse flakes value related to skin hyper-keratinization (P < 0.05)

| | | | | | | Effect | Non-effect | | Effect | | | | |
|---|---|---|---|---|---|---|---|---|---|---|---|---|---|
| No. | SNP_rs id | Chromosome | Position | Gene | Consequence | allele | allele | p-value | size | EAF_KOR | EAF_CHN | EAF_JPN | EAF_EUR |
| 1 | rs761191 | 1 | 14981920 | KAZN | intron_variant | G | A | 0.04491203 | 1.290301386 | 0.45 | 0.43 | 0.44 | 0.49 |
| 2 | rs12141342 | 1 | 15096263 | KAZN | intron_variant | A | G | 0.010813004 | 1.637090172 | 0.58 | 0.62 | 0.54 | 0.82 |
| 3 | rs79608521 | 1 | 15185050 | KAZN | intron_variant | G | A | 0.032250205 | 3.250037308 | 0.96 | 0.98 | 0.93 | 0.99 |
| 4 | rs6701221 | 1 | 152778558 | LCE1C | intron_variant | G | A | 0.034291006 | 2.434274535 | 0.91 | 0.87 | 0.91 | 0.86 |
| 5 | rs1772847 | 1 | 201278862 | PKP1 | intron_variant | C | A | 0.040928102 | 1.732610188 | 0.81 | 0.82 | 0.82 | 0.82 |
| 6 | rs832151 | 1 | 201280956 | PKP1 | intron_variant | G | A | 0.019146771 | 1.815031732 | 0.74 | 0.74 | 0.76 | 0.8 |
| 7 | rs6533497 | 4 | 111064451 | ELOVL6 | intron_variant | A | G | 0.017535562 | 3.013615051 | 0.07 | 0.09 | 0.1 | 0.43 |
| 8 | rs74874270 | 4 | 111097851 | ELOVL6 | intron_variant | A | G | 0.023188025 | 10.16947241 | 0.99 | 0.96 | 1 | 0.94 |
| 9 | rs1016835 | 6 | 7576527 | DSP | splice_region_variant, | A | G | 0.037526239 | 1.636812062 | 0.79 | 0.8 | 0.85 | 0.8 |
| | | | | | synonymous_variant | | | | | | | | |
| 10 | rs12194176 | 6 | 138413663 | PERP | intron_variant | C | A | 0.003093287 | 7.100259812 | 0.02 | 0.05 | 0.02 | 0.07 |
| 11 | rs190935218 | 11 | 64976836 | CAPN1 | missense_variant | A | G | 0.031027734 | 13.50439109 | 0 | NA | NA | NA |
| 12 | rs7304398 | 12 | 33000002 | PKP2 | intron_variant | A | G | 0.029443495 | 1.995389118 | 0.81 | 0.8 | 0.83 | 0.9 |
| 13 | rs7979825 | 12 | 33037790 | PKP2 | intron_variant | A | G | 0.004201974 | 2.873262755 | 0.1 | 0.1 | 0.08 | 0.78 |
| 14 | rs11170056 | 12 | 52628614 | KRT7 | intron_variant | G | A | 0.045138785 | 1.393599558 | 0.38 | 0.39 | 0.44 | 0.04 |
| 15 | rs1137015 | 12 | 52702681 | KRT86 | 3_prime_UTR_variant | G | A | 0.02700094 | 2.187550961 | 0.88 | 0.9 | 0.92 | 0.87 |
| 16 | rs2232398 | 12 | 52824351 | KRT75 | stop_gained | A | G | 0.02634175 | 14.30435984 | 0 | 0 | 0 | 0.01 |
| 17 | rs828367 | 12 | 53074058 | KRT1 | synonymous_variant | G | A | 0.049353475 | 12.32387471 | 0.99 | 0.99 | 1 | 0.96 |
| 18 | rs828109 | 12 | 53086626 | KRT77 | synonymous_variant | G | A | 0.049353475 | 12.32387471 | 0.99 | 0.99 | 1 | 0.96 |
| 19 | rs601072 | 12 | 53087411 | KRT77 | intron_variant | G | A | 0.049353475 | 12.32387471 | 0.99 | 0.99 | 1 | 0.96 |
| 20 | rs585664 | 12 | 53088525 | KRT77 | missense_variant | G | A | 0.049353475 | 12.32387471 | 0.99 | 0.99 | 1 | 0.96 |
| 21 | rs668313 | 12 | 53091676 | KRT77 | missense_variant | G | A | 0.049353475 | 12.32387471 | 0.99 | 0.99 | 1 | 0.96 |
| 22 | rs471122 | 15 | 43558574 | TGM5 | intron_variant | A | G | 0.029859811 | 1.438226743 | 0.57 | 0.58 | 0.56 | 0.66 |
| 23 | rs11630012 | 15 | 101875699 | PCSK6 | intron_variant | A | C | 0.025725903 | 2.563034437 | 0.07 | 0.03 | 0.1 | 0.21 |
| 24 | rs7172235 | 15 | 101955037 | PCSK6 | intron_variant | A | G | 0.015569749 | 1.936630618 | 0.81 | 0.84 | 0.79 | 0.73 |
| 25 | rs7169026 | 15 | 101965121 | PCSK6 | intron_variant | G | A | 0.02707551 | 1.76295866 | 0.79 | 0.83 | 0.8 | 0.73 |
| 26 | rs76959991 | 15 | 101970284 | PCSK6 | intron_variant | G | A | 0.041433576 | 2.976425313 | 0.93 | 0.94 | 0.93 | 0.97 |
| 27 | rs11639171 | 15 | 101987283 | PCSK6 | intron_variant | A | G | 0.043443782 | 1.504204775 | 0.29 | 0.32 | 0.3 | 0.31 |
| 28 | rs7166089 | 15 | 102028468 | PCSK6 | intron_variant | G | A | 0.025066118 | 1.565794998 | 0.31 | 0.19 | 0.27 | 0.27 |
| 29 | rs7201 | 16 | 55539614 | MMP2 | 3_prime_UTR_variant | A | C | 0.016530954 | 1.692809171 | 0.74 | 0.78 | 0.75 | 0.55 |
| 30 | rs9894356 | 17 | 8006312 | ALOXE3 | intron_variant | C | A | 0.028344123 | 3.329683661 | 0.06 | 0.04 | 0.09 | 0.27 |
| 31 | rs142219558 | 17 | 39577617 | KRT37 | splice_donor_variant | T | A | 0.024789552 | 9.997308402 | 0 | 0 | 0 | 0 |
| 32 | rs77473534 | 18 | 28602184 | DSC3 | intron_variant | G | A | 0.02602387 | 5.803415194 | 0.02 | 0.06 | 0.01 | 0.05 |
| 33 | rs1893963 | 18 | 28649042 | DSC2 | missense_variant | A | G | 0.011617191 | 3.344651958 | 0.93 | 0.92 | 0.93 | 0 |
| 34 | rs62113208 | 19 | 15652078 | CYP4F22 | intron_variant | A | G | 0.0385653 | 9.265979715 | 1 | 0.99 | 0.96 | 0.78 |
| 35 | rs118065021 | 19 | 34250852 | CHST8 | intron_variant | A | T | 0.037934898 | 2.679562257 | 0.92 | 0.04 | 0.05 | 0 |
| 36 | rs11083944 | 19 | 49072265 | SULT2B1 | intron_variant | G | A | 0.000336803 | 2.581162835 | 0.28 | 0.3 | 0.27 | 0.52 |
| 37 | rs8111787 | 19 | 49073974 | SULT2B1 | intron_variant | A | G | 0.000272279 | 2.652606591 | 0.28 | 0.3 | 0.27 | 0.66 |
| 38 | rs2544796 | 19 | 49075277 | SULT2B1 | intron_variant | G | A | 0.000272279 | 2.652606591 | 0.28 | 0.3 | 0.27 | 0.66 |
| 39 | rs6509394 | 19 | 49075781 | SULT2B1 | intron_variant | G | A | 0.000272279 | 2.652606591 | 0.28 | 0.3 | 0.27 | 0.66 |
| 40 | rs3826827 | 19 | 49076923 | SULT2B1 | intron_variant | C | G | 0.000505149 | 2.536227581 | 0.28 | 0.28 | 0.27 | 0.52 |
| 41 | rs3760804 | 19 | 49077057 | SULT2B1 | intron_variant | A | G | 0.017873537 | 1.808506265 | 0.74 | 0.68 | 0.71 | 0.84 |
| 42 | rs58265481 | 19 | 49087798 | SULT2B1 | intron_variant | C | G | 0.044085926 | 3.418883149 | 0.08 | 0.07 | 0.1 | 0.16 |
| 43 | rs12460535 | 19 | 49098750 | SULT2B1 | intron_variant | A | G | 0.020167421 | 1.686709328 | 0.51 | 0.6 | 0.54 | 0.36 |
| 44 | rs60863739 | 19 | 51481876 | KLK7 | intron_variant, | A | C | 0.015275718 | 5.820478799 | 0.02 | 0.02 | 0.06 | 0.09 |
| | | | | | NMD_transcript_{—} | | | | | | | | |
| | | | | | variant | | | | | | | | |
| 45 | rs3745535 | 19 | 51520487 | KLK10 | missense_variant | A | C | 0.01750634 | 1.579867954 | 0.4 | 0.32 | 0.41 | 0.38 |
| 46 | rs214782 | 20 | 2281970 | TGM3 | intron_variant | G | A | 0.014498986 | 3.266747408 | 0.07 | 0.08 | 0.05 | 0.16 |
| 47 | rs214803 | 20 | 2290333 | TGM3 | missense_variant | C | A | 0.014498986 | 3.266747408 | 0.07 | 0.09 | 0.05 | 0.16 |

### Example 1: Genotypic Polymorphism of Rs10502560 and Confirmation of Improvement in Turnover Rate by Serine Prescription

Difference in the phenotypes and skin turnover rate improvement effect were compared according to the genotypes of rs10502560 which is a representative example discovered as a single nucleotide polymorphism (SNP) marker with correlation significance with skin turnover and hyper-keratinization.

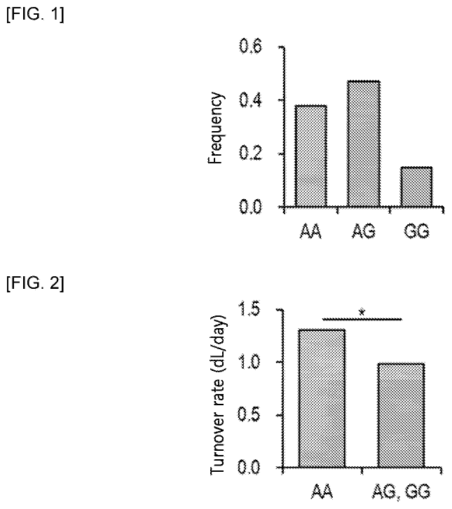

In detail, the genotypes of rs10502560 show various frequencies in Koreans. For example, the genotypes of rs10502560 were confirmed to have AA frequency of 0.38, AG frequency of 0.47, and GG frequency of 0.15 (FIG. 1).

The AG or GG genotype with G which is the effect allele of rs10502560 was designated as a case group, and the AA genotype without the effect allele G was designated as a control group. 34 people were divided into the control group and the case group, and the average turnover rates were compared to confirm that the turnover rate of the case group was significantly slower than that of the control group (FIG. 2). Specifically, it was confirmed that the turnover rate (dL/day) of the control group (AA) was 1.3, and the turnover rate (dL/day) of the case groups (AG, GG) was 1.0.

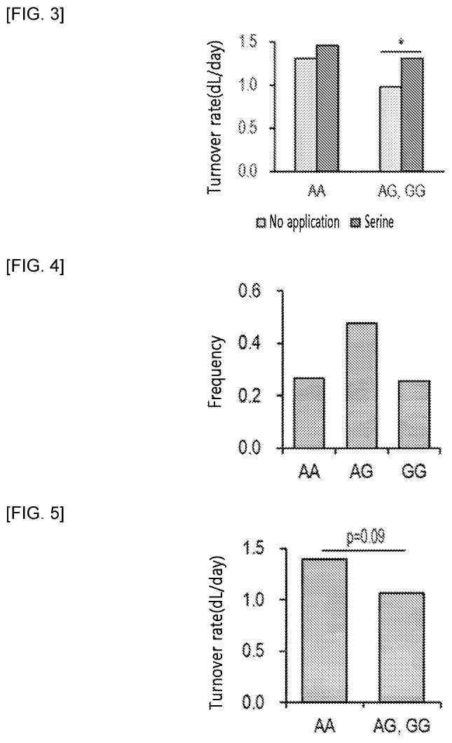

Based on the rs10502560 genotypes, the control group (genotype AA) and the case group (genotype AG or GG) were classified, and a formulation containing 5% of serine, which is a turnover enhancing material, was prescribed to be used once a day. With respect to each group, the turnover rates were compared between the non-application area and the serine-containing formulation application area. In the control group, the average turnover rate of the non-application area was 1.31 and the average turnover rate of the serine application area was 1.46, indicating that an increase was approximately 11%, but difference in the values was not significant. In the case group, the average turnover rate of the non-application area was 0.98 and the average turnover rate of the serine application area was 1.31, indicating that an increase was approximately 34% and difference in the values was significant (FIG. 3).

These results show that the turnover rate of an individual may be determined through the rs10502560 genotypes, and the slow turnover rate of the case group may be improved through the use of the turnover enhancing material (skin care products), as compared to the control group.

### Example 2: Genotypic Polymorphism of Rs16853334 and Confirmation of Improvement in Turnover Rate by Protease Prescription

Difference in the phenotypes and skin turnover rate improvement effect were compared according to the genotypes of rs16853334 which is a representative example discovered as a single nucleotide polymorphism (SNP) marker with correlation significance with skin turnover and hyper-keratinization.

In detail, the genotypes of rs16853334 show various frequencies in Koreans. For example, the genotypes of rs16853334 were confirmed to have AA frequency of 0.27, AG frequency of 0.48, and GG frequency of 0.25 (FIG. 4).

The AG or GG genotype with G which is the effect allele of rs16853334 was designated as a case group, and the AA genotype without the effect allele G was designated as a control group. 34 people were divided into the control group and the case group, and the average turnover rates were compared to confirm that the turnover rate of the case group was slower than that of the control group (FIG. 5). Specifically, it was confirmed that the turnover rate (dL/day) of the control group (AA) was 1.4, and the turnover rate (dL/day) of the case groups (AG, GG) was 1.1.

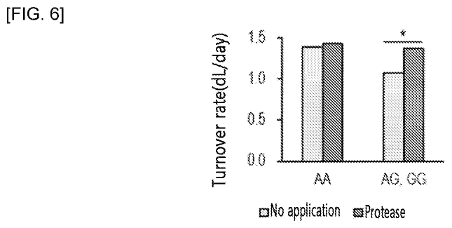

Based on the rs16853334 genotypes, the control group (genotype AA) and the case group (genotype AG or GG) were classified, and a formulation containing 5% of protease (brand name: Keratinase H, LCS Biotech Co., Ltd.), which is a turnover enhancing material, was prescribed to be used once a day. With respect to each group, the turnover rates were compared between the non-application area and the protease-containing formulation application area. In the control group, the average turnover rate of the non-application area was 1.39 and the average turnover rate of the protease application area was 1.43, indicating that an increase was approximately 3%, but difference in the values was not significant. In the case group, the average turnover rate of the non-application area was 1.07 and the average turnover rate of the protease application area was 1.37, indicating that an increase was approximately 28% and difference in the values was significant (FIG. 6).

These results show that the turnover rate of an individual may be determined through the rs16853334 genotypes, and the slow turnover rate of the case group may be improved through the use of the turnover enhancing material (skin care products), as compared to the control group.

Based on the above description, it will be understood by those skilled in the art that the present invention may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1-10. (canceled)

11. A method of providing information for determining skin turnover and hyper-keratinization, the method comprising the steps of:
(a) amplifying a polymorphic site of the single nucleotide polymorphism marker for determining skin turnover and hyper-keratinization of selected from Table 1 to Table 3 in DNA obtained from a sample isolated from a subject or hybridizing the same with a probe; and
(b) identifying a base of the amplified or hybridized polymorphic site of the step (a).

12. The method of claim 11, wherein the sample is hair, urine, blood, various body fluids, isolated tissues, isolated cells, or saliva.

13. The method of claim 11, wherein the amplification and identification of the polymorphic site are performed using a SNP chip.

14. A method of controlling skin turnover and hyper-keratinization, the method comprising the steps of:
(a) amplifying a polymorphic site of the single nucleotide polymorphism marker for determining skin turnover and hyper-keratinization of selected from Table 1 to Table 3 in DNA obtained from a sample isolated from a subject or hybridizing the same with a probe;
(b) identifying a base of the amplified or hybridized polymorphic site of the step (a); and
(c) prescribing skin care products when the base of the amplified or hybridized polymorphic site includes one or more of bases that are effect alleles according to any one of Table 1 to Table 3.

15. A composition for determining skin turnover and hyper-keratinization, the composition comprising a probe capable of detecting any one or more single nucleotide polymorphism (SNP) markers for determining skin turnover and hyper-keratinization selected from Table 1 to Table 3 or an agent capable of amplifying the same.

16. The composition of claim 15, wherein the single nucleotide polymorphism markers for determining skin turnover and hyper-keratinization further includes one or more polynucleotides selected from the group consisting of polynucleotides corresponding to one or more single nucleotide polymorphism markers selected from any one of Table 1 to Table 3; and complementary polynucleotides thereof.

17. The composition of claim 15, wherein the determining of skin turnover and hyper-keratinization is determining of the skin turnover rate and hyper-keratinization.

18. A kit for determining skin turnover and hyper-keratinization, the kit comprising the composition of claim 15.

19. A kit for determining skin turnover and hyper-keratinization, the kit comprising the composition of claim 16.

20. The kit of claim 18, wherein the kit is an RT-PCR kit or a DNA chip kit.

21. The kit of claim 19, wherein the kit is an RT-PCR kit or a DNA chip kit.

22. A microarray for determining skin turnover and hyper-keratinization, the microarray comprising the single nucleotide polymorphism (SNP) markers for determining skin turnover and hyper-keratinization of claim 15.
